(19) United States
(12) Patent Application Publication
(10) Pub. No.: US 2004/0148675 A1
(43) Pub. Date: Aug. 5, 2004

(54) Utility glove

(76) Inventor: Michael Charlton Powell, Tring Herts (GB)

Correspondence Address: BIRCH STEWART KOLASCH & BIRCH, PO BOX 747, FALLS CHURCH, VA, 22040-0747, US

(21) Appl. No.: 10/476,414

(22) PCT Filed: Apr. 26, 2002
(86) PCT No.: PCT/EP02/04643

(30) Foreign Application Priority Data
May 1, 2001 (GB) 0110649.1

(51) Int. Cl. A41D 13/08; A61F 9/00

(52) U.S. Cl. 2/16

(57) ABSTRACT

There is provided a glove-like interface for use in protecting a user's hand during utility tasks comprising a body having a plurality of finger members extending therefrom; and resilient hand support means for securing the glove to a user's hand, wherein the resilient hand support means comprises both finger support means and palm support means for respectively securing the glove to the fingers and palm of the user's hand.

### TECHNICAL FIELD

The present invention relates to a utility glove. In particular, the present invention relates to a glove for receipt of utility or tool attachments, especially attachments suitable for use in cleaning and grooming applications.

### BACKGROUND TO THE INVENTION

Most of the cleaning tools presently available rely on a user directly holding an independent interface, such as handle of the common bristle brush. In such cases, the user's hand is unprotected whilst conducting the task. Additionally, much of the innate dexterity of the user's hand, in particular, the user's fingers, is lost.

It is an object of the present invention to provide a glove-like (i.e. wearable by the hand) hand-utility tool interface, which protects the hand of a user during the undertaking of various utility tasks (e.g. cleaning, grooming).

It is another object of the present invention to provide such a glove-like interface, which affords the user more of the dexterity of the user's hand, such that the interface can form itself to meet any surface, regardless of its complexity.

It is a further object of the present invention to provide a glove-like interface, which allows the hand to be clenched, for example, for wringing out liquid from the glove-like interface, without causing discomfort to the wearer.

It is a still further object of the invention to provide a glove-like interface, which is adaptable for use with different sizes of hand and different lengths of finger.

### SUMMARY OF THE INVENTION

According to one aspect of the present invention there is provided a glove-like interface for use in protecting a user's hand during utility tasks comprising a body having a plurality of finger members extending therefrom; and resilient hand support means for securing the glove to a hand, wherein said resilient hand support means comprises both finger support means and palm support means for respectively securing the glove to the fingers and palm of a user's hand.

The term ‘glove-like interface’ (or simply ‘glove’) herein is used to mean a wearable framework, device or apparatus, which protects at least part of the hand of a user during the undertaking of various utility tasks (e.g. cleaning, grooming). The ‘glove’ provides an interface between the user's hand and the utility media, which in aspects comprises a utility tool or utility media (e.g. cleaning or polishing media) for use in performing a utility task.

In aspects, the glove is designed to affords maximum comfort to the hand whilst maximising the work area covered by the hand and the efficiency of the cleaning task.

Suitably, the glove is adaptable for use with user's hand's of varying sizes.

In one aspect, the glove is a full glove (i.e. all of the hand and fingers are enclosable thereby).

In another aspect, the glove is a part-glove (i.e. at least part of the hand and fingers are not enclosable thereby). Aspects are envisaged in which the glove has an open framework or lattice-type structure.

Suitably, the glove-like interface in use, covers the users' fingers and at least part of the user's palm but leaves the top part of the user's hand and fingers uncovered. This embodiment provides enhanced user utility in terms of ease of hand and finger movements.

In embodiments, the glove-like interface herein is also shaped to be put on by the user in a one-handed operation which enhances ease and speed of user “pick up”. This contrasts with the operation to put on most traditional gloves, which is a two handed operation.

The glove-like interface is provided with a body, which in use, typically covers part or all of the user's palm and fingers. The body has a plurality (e.g. four) of finger members extending therefrom. In aspects, a thumb member is also provided.

The body has essentially two functions. Firstly, it provides a degree of protection to the user's hand whilst conducting utility tasks. Secondly it provides a support for the application of utility media by the hand.

Suitably, the body provides a rigid support for the utility media, but is entirely flexible to the movement and dexterity of the hand. In one aspect, the body provides a rigid mounting for the attachment of utility attachments thereto.

Suitably, the body is shaped to provide a uniform, e.g. flat work surface (i.e. utility surface). This contrasts with the non-uniform, rounded form, work surface provided by the un-gloved palm and underside of fingers of a user's hand.

In aspects, the body is shaped to improve overall glove flexibility. In aspects, the palm-protecting part of the body is shaped and/or cut with various patterns. In one aspect, a split is provided deep into the palm element in the line between each finger element to allow maximum lateral flexibility regardless of the size of hand being applied to it. The split lines conclude in a “T” form, which further enhances lateral flexibility.

In one aspect, the finger members incorporate a feature at the junction thereof with the palm element, which adapts to account for extremes of finger length and optionally, enhances the framework's lateral dexterity. Suitably, this feature comprises a diamond-shaped extendable element.

In one aspect, the finger members have side walls to at least partially encase a finger. The side walls do not necessarily have a finger support function, although embodiments are envisaged in which the side walls do from components of the finger support means herein.

In one aspect, the glove is designed such that it covers only that part of the palm that can be comfortably reached by the fingers. When the glove comprises absorbent media, this provides the function that the glove can be efficiently wrung out by a simple clenching of the fist.

Optionally, the glove is provided with an ancillary interface such as in the form of a flexible backing sheet. This ancillary interface may be shaped and sized such as to enhance the coverage of the user's hand. The ancillary interface may also be provided with attachment means (e.g. in the form of snap-fit contourings) for attachment to the glove.

Suitably, the glove-like interface herein is arranged to cooperate with those elements common to all sizes of user hands. Without reliance on fabric elasticity, it is suitably configured to offer a fit for the majority (e.g. 90%+) of all adult hand sizes. Traditional glove forms account for this variable by offering different sizes of product or by use of stretch fabrics, whereas this interface does so by means of resilient hand support means for securing the glove to a user's hand.

The resilient hand support means comprises both finger support means and palm support means for respectively securing the glove to both the fingers and palm elements of a user's hand. The support may in aspects have a gripping function (i.e. finger grip means and/or palm grip means). Preferably, the glove comprises finger support means on each finger member.

The resilient finger support means acts to hold the glove tight against the underside of at least part of the user's fingers. Suitably, this prevents any tendency for the glove to sag downwards from the fingers. In aspects, the resilient finger support means also acts to hold the glove firmly up against the user's finger-tips.

Given that the finger support means are resilient, they can adjust to the thickness of the finger and hence accommodate fingers of different length and size. The resilience may come from the material of construction of the support means (i.e. the material itself is resilient) or it may come from the structural form/arrangement of the support means e.g. through play or springing.

In one aspect, the resilient finger support means comprises a caged framework (e.g. housing) shaped to snugly receive a user's finger tip. The cage provides ample space for a user's finger-nail.

In another aspect, the resilient finger support means defines a constricted passageway shaped to engage with a user's finger. Suitably, the constriction is of resilient character such that a first finger joint may be squeezed into the passageway past the constriction. In this case, the resilient finger support means typically secures a user's finger between the first and second joints of that finger.

In one aspect, the resilient finger support means takes the form of a sprung clip, which itself may act to constrict a finger passageway.

In another aspect, the passageway comprises side walls provided with grips for gripping above and beside the finger between its first and second joints, so securing the framework to each finger. Suitably, at the point where these grips act on the side wall, the inner face of the wall is “bellied” inwards, better to grip the natural form of the finger. Suitably, these “bellied” forms are provided with a “saw tooth” form to better grip the finger.

In one aspect, the finger grips are provided with side or top mounted lugs, which are shaped to cooperate with a base station (described hereinafter).

The palm support means herein functions to support the body (e.g. palm-protecting part) of the glove and to retain it firmly against the underside of the user's hand. Suitably, the palm support means secures the body of the glove to the base of the fingers and/or the palm of the user's hand.

Suitably, the palm support means takes the form of one or more resilient yokes. The yokes are shaped for receipt at the junction between the fingers of a user (i.e. at the junction of either the first and second, second and third, or third and fourth fingers).

Suitably, the yokes are shaped to cooperate with the natural taper common to all hands. The resilient nature of the yoke once again affords the glove to accommodate and adjust to various hand sizes.

Suitably, the one or more yokes have a T-form. In particular, each yoke comprises a vertical trunk with horizontally extended arms and legs. In use, the T-form yoke fits between a user's adjacent fingers.

In one aspect, each yoke comprises two pairs of independent arms, each pair mounted on a corresponding vertical trunk. This configuration allows even greater freedom of movement.

Suitably, in use with a large hand the vertical trunk of the yoke sits forward of the junction of finger and palm of a large hand, so that in use, the shorter fingers of a smaller hand may engage fully with the finger support means.

Preferably, the glove comprises two or three resilient palm support members (e.g. yokes), each of which may comprise pairs of independent arms.

In aspects, the resilient finger and/or palm support means are made from sprung steel wire or moulded (e.g. injection moulded) plastics.

In one aspect, the glove additionally comprises at least one substantially “V”-shaped excision running transversely across the underside of the glove, preferably on the finger members. The combination of the flexibility of the glove material and the presence of the “V”-shaped excisions permits the glove to collapse with ease, for example, if the wearer clenches his/her fist, e.g. to wring any liquid from the glove.

Suitably, between each “V”-shaped excision there is provided a bridge member. Each bridge member may form a support for the utility attachments on the underside of each finger member. When the hand is clenched into a fist, the combined width of the one or more bridge members may approximate to the reduced length of the underside of the finger member. Thus, there is no “bulking” of glove material, for example, inside a clenched fist.

Suitably, the glove has more than one substantially “V”-shaped excision running transversely across the underside of each finger member. Suitably, the glove has a plurality of “V”-shaped excisions running transversely across the underside of each finger member. Typically, each “V”-shaped excision is separated by a bridge member.

In a subsidiary aspect, the presence of “V”-shaped excisions on the underside of the glove can also facilitate the accommodation of different hand sizes. This is because the incisions render the finger members highly flexible so that an over wide finger may simply displace them.

Suitably, the glove is made from a semi-rigid or rigid material. In aspects, polymer materials such as those suitable for use in injection moulding processes are suitable.

Suitably, parts of the glove are constructed from fabrics and/or composite materials.

Suitably, the glove additionally comprises means to receive one or more utility attachments. As used herein, the term “utility attachments” refers to attachments having a useful function. For example, the utility attachments may take the form of tools for any useful purpose.

Suitable purposes include cleaning; grooming; DIY purposes including sanding, painting, shaping and forming; medical purposes; patient care purposes such as bed bathing; medical aids for arthritis and other complaints of the hands; surgical applications, including “remote managed operations; sports purposes, including with webbing sheet between the fingers, a speed swimming aid (i.e. a “hand-flipper”); automotive car care including washing, polishing, leathering and interior cleaning; health and beauty purposes such as ex-foliation, massage, application of oils/lotions; gardening purposes such as plant care, house plant leaf cleaning/treatment; shoe and boot polishing; window cleaning; security purposes including forms with electronic means included for body searching and the like; military applications such as versions carrying electronic means for added safety in bomb disposal and mine clearing; versions with micro cameras attached so that students may see clearly the fine detail of manual tasks performed by their tutors; grooming of humans and of animals. Indeed, the glove-like structure may find utility in pretty much any and every task managed by hand.

The utility attachments may either be permanently attached to the glove or reversibly receivable by the glove. For example, the glove-like interface and utility attachment may be formed as cooperating elements, the utility attachment being interchangeable and or replaceable.

The utility attachments may take the form of bristles, spines, hooks, hair, sponge, leather, fabric pads, scourers, abrasives or wire wool.

The utility attachments may also comprise vacuum elements.

The means to receive utility attachments may be positioned at the front and/or underside and/or sides of said finger members.

In one embodiment, the glove may further comprise means to receive utility attachments on the body of the glove.

The means to receive utility attachments may comprise one or more circular recesses. These may provide the means to locate cleaning media and provide the home, for example, for bristle bunches.

Suitably, the means to receive utility attachments may comprise at least one female socket for receiving a male counterpart situated on a utility attachment.

In one vacuum aspect, a resilient ‘fish-tail’ nozzle attaches to the under side of the glove. Leading therefrom a very light weight hose goes back, under the wrist and is supported mid way between wrist and elbow by a loop or hook form which goes over the arm. Suitably, a flexible membrane covers the underside of the glove, allowing the fingers to be spread without loss of vacuum between the fingers. Further, this flexible membrane extends to provide a “skirt” positioned outside the bristles, running around the outside of the palm and up the outside of the index and little finger, once again preventing loss of suction and promoting that the vacuum is available under the finger tip bristles. The bristle part of this particular tool is in the form of a flexible membrane with bristles lining the side of each finger and, surrounding the underside of the glove. The vacuum then draws down the bristle “avenue” underneath each finger, and generally around the palm.

In another aspect, the glove incorporates means for using liquids and gasses as “tools”, both flowing out through and being drawn in through attachable media. A reservoir for dispensing such gasses and or liquids may be additionally incorporated as an integral or separate, but connected, feature of the claimed invention.

In one embodiment, the utility attachments are permanently attached to the glove.

In another embodiment, the utility attachments are removable and/or exchangeable.

In one aspect, the glove and/or utility attachments have electrical and/or electronic capability. In one example, the glove-like interface itself is configured to incorporate said electrical or electronic components, rather than them being a separate utility attachment.

In a particular example, the glove-like attachment is adapted to perform the function of a computer mouse. In another example, a pair of gloves acts such as to replace both the mouse and key board of a conventional computer. In such instance, the key board might become a mere projection onto the desk or other surface.

In one embodiment, the form of the glove is intended to co-operate with a “round finned bar”. Squeezing the bar in the fist of the glove allows wringing out of absorbent cleaning media, by squeezing the bar in the grip. This enables the detritus and liquid carried in/on the glove to be efficiently expelled, down the channels provided between the fins. In aspects, the bar is arranged to release disinfectant in each such squeezing. This may be utilised to further enhance the cleaning performance of the product.

In another aspect, the invention provides a housing for said glove as defined supra, said housing comprising a plurality of finger channels to receive said finger members therein.

Preferably, said finger channels taper such that when the glove is in situ, said finger support means are urged open. In this embodiment, a user may insert his/her hand into the glove with ease. By lifting the hand away from the housing, the glove is immediately secured onto the hand as the finger support means tighten about the fingers of the hand.

Typically, the glove is maintained in the housing by the frictional forces between the glove material and the resilient force of the finger support means.

Preferably, the housing is wall mounted to keep any work surfaces free from clutter.

Suitably, the housing is of sufficient depth to accommodate the glove and any utility attachments attached thereto.

In one aspect, the finger elements and/or finger support means of the glove are provided with side or top mounted lugs, which are shaped to cooperate with a base station. Suitably, the base station is in this aspect provided outward bowed mounting rails such that, when the hand pushes the framework into the base station, the finger grips are caused to open, so releasing the fingers and “parking” the glove. The glove is automatically coupled to the hand again by inserting the fingers and driving it forward off the rails.

In another aspect, the base station is arranged to interact with the glove such that the whole of a finger-tip assembly is opened by its interaction with the base station.

This embodiment provides for the efficient gripping of all sizes of finger-tip.

In other aspects, the invention separately provides a glove; a housing; and utility attachments for use with the glove as described supra.

In another aspect, the invention provides a set of utility attachments for use with the glove as defined supra.

In yet another aspect, the invention provides a kit of parts for a utility glove comprising a glove as defined supra and a set of utility attachments as defined supra. Optionally, the kit of parts further comprises a housing for said glove.

In another aspect, the invention provides finger support means for use with the glove described herein. Suitable resilient finger support means comprises a caged framework (e.g. housing) shaped to snugly receive a user's finger tip; and a constricted passageway (e.g. formed by a loop or by gripping arms) shaped to engage with a user's finger.

In aspects, the finger support means is joined to the glove-like interface by a loop running around the finger, which in use locates approximately at the back of the user's finger nail. This provides the only connection with the interface. Back (palmwards) the finger gripping “arms” clamp inwards against the material of the interface, but are not attached to it. This permits free movement between these two elements, which allows for fist clenching without causing discomfort through constricting the finger. Essentially, in clenching, the finger becomes “deeper” north to south, and so requires that the assembly may easily expand at this point to account for it.

The caged framework of the finger support means provides ample space for a user's finger-nail. The constriction of the passageway is of resilient character such that a first finger joint may be squeezed into the passageway past the constriction. In this case, the resilient finger support means typically secures a user's finger between the first and second joints of that finger.

In one aspect, the resilient finger support means comprises a sprung clip, which itself may act to constrict a finger passageway.

In another aspect, the passageway comprises side walls provided with grips for gripping above and beside the finger between its first and second joints, so securing the framework to each finger. Suitably, at the point where these grips act on the side wall, the inner face of the wall is “bellied” inwards, better to grip the natural form of the finger. Suitably, these “bellied” forms are provided with a “saw tooth” form to better grip the finger.

In one aspect, the finger grips are provided with side or top mounted lugs, which are shaped to cooperate with a base station (described hereinbefore).

### BRIEF DESCRIPTION OF THE DRAWINGS

The invention will now be described further with reference to the accompanying drawings, in which:—

FIG. 1 shows a glove in accordance with the invention including the finger and palm support means;

FIG. 2 shows the glove of FIG. 1 in the absence of finger and palm support means;

FIG. 3 shows finger support means in accordance with the present invention;

FIG. 4 shows palm support means in accordance with the present invention;

FIG. 5 shows a housing or base station for the glove in accordance with the present invention;

FIG. 6a shows a side view of a second glove in accordance with the invention;

FIG. 6b shows a top cross-sectional view of the second glove of FIG. 6a shown along the cross-section defined by line A-A′ of FIG. 6a;

FIG. 6c shows a back end elevation of the second glove of FIG. 6a;

FIG. 6d shows palm support means suitable for use in the second glove of FIG. 6a;

FIG. 7a (which corresponds essentially to element 112d of FIG. 6a) and FIG. 7b show the docking interaction between the finger support means of a glove (shown in FIG. 7a in top view) with a parking station (shown in FIG. 7b in top plan view) of a base station;

FIGS. 8a and 8b show a finger tip cage, wherein the whole of the cage opens and closes about the finger tip and wherein the cage has top-mounted lugs; FIG. 8b also shows in plan view a wedge feature of a base station required to open the finger tip cage; and

FIGS. 9a and 9b respectively show side and lengthways cross-sectional views of a finned bar shaped for grip by a glove herein.

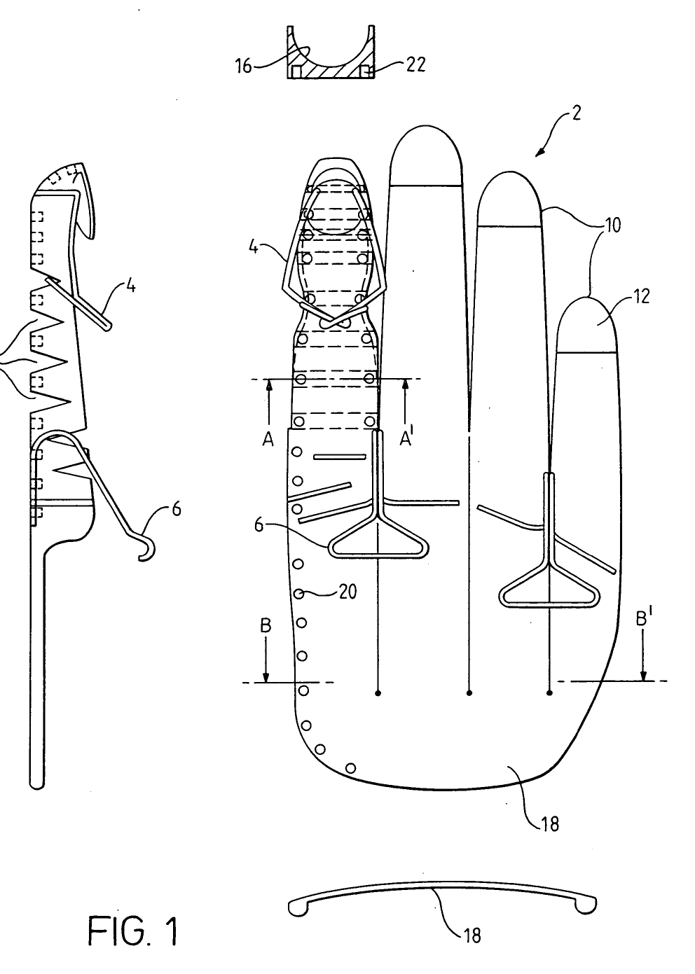

Referring now to the drawings, FIG. 1 illustrates a glove 2 having a finger grip 4 and a palm yoke 6 assembled together.

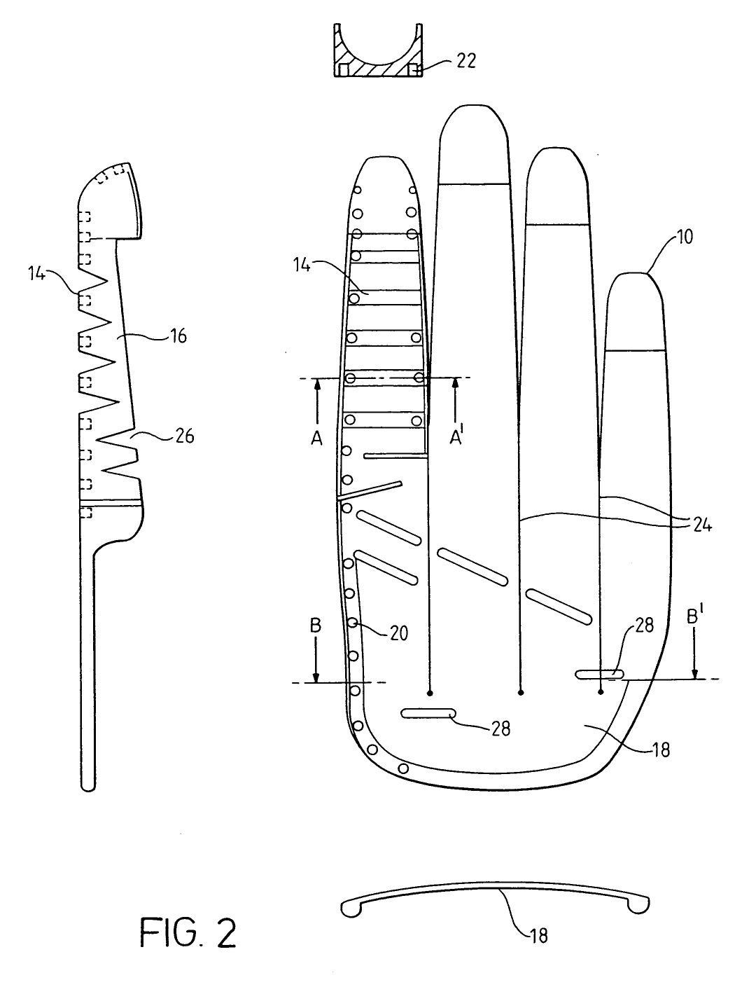

FIG. 2 illustrates the glove 2 following its injection moulding before the attachment of the finger grip 4 and palm yoke 6.

The glove 2 is essentially in the form of a part-glove which in use, will cover the underside of the user's four fingers and palm and the tips of each finger. The glove has four finger-like extensions 10, each extension having a thimble-like end 12 to house a finger-tip, and having space for a finger-nail. Each finger-tip end 12 has the means to accommodate utility attachments such as cleaning media on the front, sides and underside.

The glove 2 is moulded from mildly flexible plastics. The combination of the flexibility of the base material and the “V”-shaped excisions 8 made in the underside of the glove and which run transversely across each finger-like extension 10, permit its easy collapse inside a clenched fist.

Between each “V”-shaped excision is a bridge 14 of glove material which forms the support for the cleaning attachments (not shown) on the underside of each finger-like extension 10. When the fist is clenched, the combined width of the bridges 14 approximates to the reduced length of the underside of the finger-like extension 10.

In this example, the side walls 16 of the finger-like extensions 10 are designed to rise to about half the depth of an average adult finger.

The bridges 14 and the body 18 of the glove are provided with circular recesses 20 to provide a means to locate the cleaning media (not shown), that is, they provide the home for, for example, bristle bunches. Such recesses 20 may be incorporated into other locations across the body 18 and finger-like extensions 10 of the glove 2.

The required strength and resilience of the cleaning media once attached, is provided under the finger-like extensions 10 by the recesses 20 being set in the thickest sections 22 of the injected material.

The body 18 of the glove 2 is designed to cover only that part of a palm that can be comfortably reached by the fingers once they are curled over. This ensures that absorbent cleaning media can be efficiently wrung out by a simple clenching of the fist. Suitably, the palm piece of the body 18 also sized to represent the size of a small user hand's palm (i.e. it is as big as it can be made without it going beyond the palm of a small user hand's palm). As such, it contributes to the utility of making one size of glove fit all. Other types of media may be attached to other parts of the glove and extend to the periphery of the glove body 18 and/or to the area covered by the whole hand.

To facilitate the accommodation of a particularly large hand, the dividing line 24 between each finger-like extension 10 extends far into the body 18 of the glove.

At the base of each finger-like extension 10, the glove has two opposing cuts 26. These cuts 26 ease the clenching of a user's fist and provide flexibility in accommodating an unusually long user's finger.

The body 18 may be provided with narrow apertures located in the finger line to ease the collapse of the body and assist in the body's conformation to different shapes.

At the back of the glove body 18 are two further apertures 28 which are designed to be slipped over the yoke pieces 6 so that their hook like nature is eliminated for the purpose of machine washing the glove.

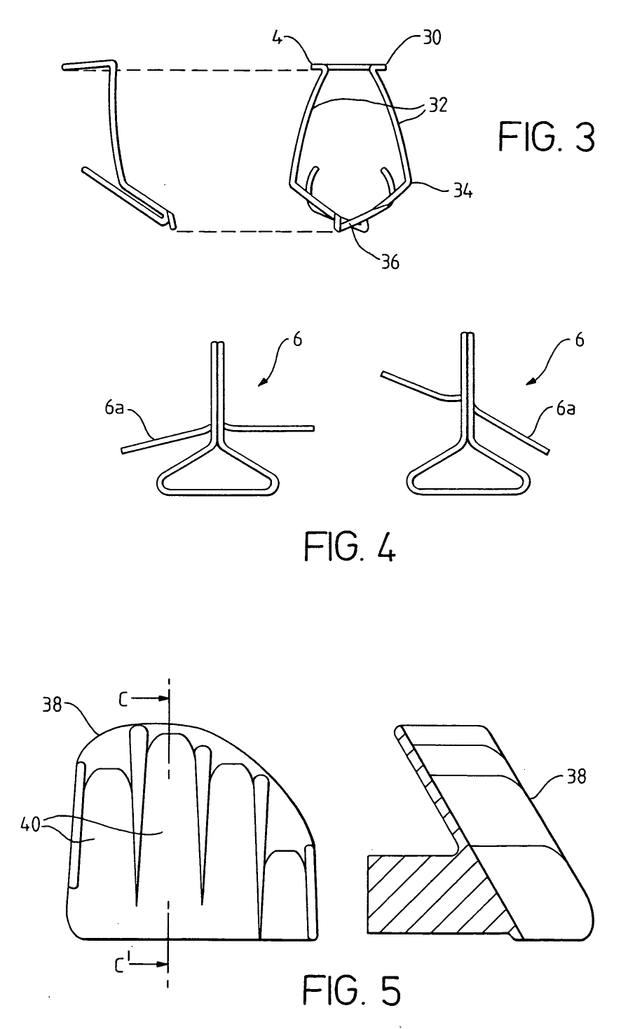

The finger grip 4 is further explained in FIG. 3. Each finger-like extension 10 has a finger grip 4.

The finger grip 4 is made form sprung steel wire or moulded plastics and fitted to the glove after the glove is injection moulded. The rounded portion 30 where its two arms 32 meet extends over the finger and snaps into a channel provided for it (not shown).

From the rounded portion 30 the two arms 32 bow outward. At the shoulder point 34 they are pressed inwards towards each other. Thus, the end points of each arm, the gripping portions 36, are forced to part so permitting the first knuckle of even a large hand to pass easily through.

If the inward pressure on the shoulders 34 is released, the grips 4 are biased to resume their original positions and so exert a gripping inward pressure on each side of the finger.

As the pressure is exerted against the side walls 16 of the glove 2, the finger grips 4 grip the finger comfortably.

The gripping action usually takes place between the first and the second joints of a finger. This location on the finger can take significant pressure without discomfort.

The gripping point will differ for different hand sizes. On a small hand, the grip is more or less central to the first and second finger joints; on a large hand the grip will occur just behind the first finger joint.

The side view of the finger grip 4 shows that the gripping portion of the arms 32 is angled so that it grips the finger diagonally. This feature allows the user's fist to be clenched for wringing out an absorbent media whilst not presenting discomfort. A diagonal grip also contributes to efficiency of the gripping force and its capacity to cope with a wide range of different finger sizes.

FIG. 4 illustrates the palm support means or yokes 6. The function of the yokes 6 is to support the main part of the glove 2 and to hold it firmly against the underside of the user's hand.

The yokes 6 are made from sprung steel wire or are formed in plastics. The yokes 6 are fitted to the glove following injection moulding. A yoke 6 fits into a recess (not shown) formed in the underside of the glove and it protrudes upwards between the index finger and the adjacent finger. Another yoke protrudes between the third and the fourth finger.

In another embodiment there may be three yokes, one rising in between each two fingers. By way of alternative, one or more of the yokes may be replaced by a loop of glove material. There also may be a loop of glove material extending around the whole of the palm of the hand.

The descending parallel wires 6a of each yoke are shown in the ‘in use’ position in which they touch one another (i.e. pressing the wires together against their memory).

In the ‘at rest’ position the descending parallel wires will part leaving a narrow (e.g. ⅔ mm) gap at the upper narrowest point then diverging very slightly as they descend.

In the side elevation shown in FIG. 1 it can be seen that the yokes 6 are tapered towards the finger tip. This tapering form is designed to provide good support regardless of the size of hand. When the user's finger-tips are tightly inserted into the glove, the webbed skin between the fingers of a small hand will be touching the narrowest part of the taper. For a large hand, there will be a gap between that part of the yoke and the webbed skin. Thus, a small hand proceeds further into the taper than a large hand.

Further aspects of the utility of the yokes of the glove in accommodating hands of differing sizes may be understood by the following description:

In section, the fingers of a user themselves taper. In their length, the fingers of a user also taper. In depth, the hand and fingers form a taper from wrist to fingertip. The glove herein takes advantage of this commonality by providing that a small hand may progress deeper into the glove's taper than may a large hand, so providing that whatever the size of hand, the finger tips may always reach the glove's fingertips.

The glove herein thus gives freedom for the fingertips of a small hand to reach the glove's fingertips, by eliminating the “stop” material of the traditional glove form, which rises between the fingers where they join the body of the hand. Having eliminated that material “stop”, (and so lost the support that is provided by the stop continuing up to encircle the finger), the glove provides a replacement support in the form of the yokes as described herein.

The full extent of the yoke's taper is set to be sufficiently forward that short fingers may reach the fingertips of the glove.

These yokes take advantage of the hand's tapering form. A small hand is less deep at the point where fingers join the main body of the hand, and so, at its most forward point, the yoke tapers downwards to provide a snug fit for the small hand. Whereas, the wider top portion of the yoke's taper provides a snug fit for the considerably greater depth of a big hand. The taper between these extremes accommodates all of the in-between hand sizes.

Having descended to its lowest point, the yoke members then return back towards the palm before they make their connection to the interface. This return provides that their connection to the interface is beneath the junction of finger and palm of a large hand, so not to impede its full dexterity. Whereas, for the small hand, the connection is slightly under the palm where it will cause no obstacle to dexterity.

Returning to a description of the drawings, the housing 38 is shown in FIG. 5 and has four finger channels 40, one for each finger-like extension on the glove 2. The channels 40 taper towards the top. The housing 38 has two functions; firstly, it provides a storage place for the glove. Additionally, it facilitates the application of the glove 2 to the hand of a user; when in situ in the housing, the shoulders 34 of each finger grip 4 are constricted within the tapering finger channels 40, thus urging the grips 4 open and so easing the insertion of the fingers into the finger-like extension 10.

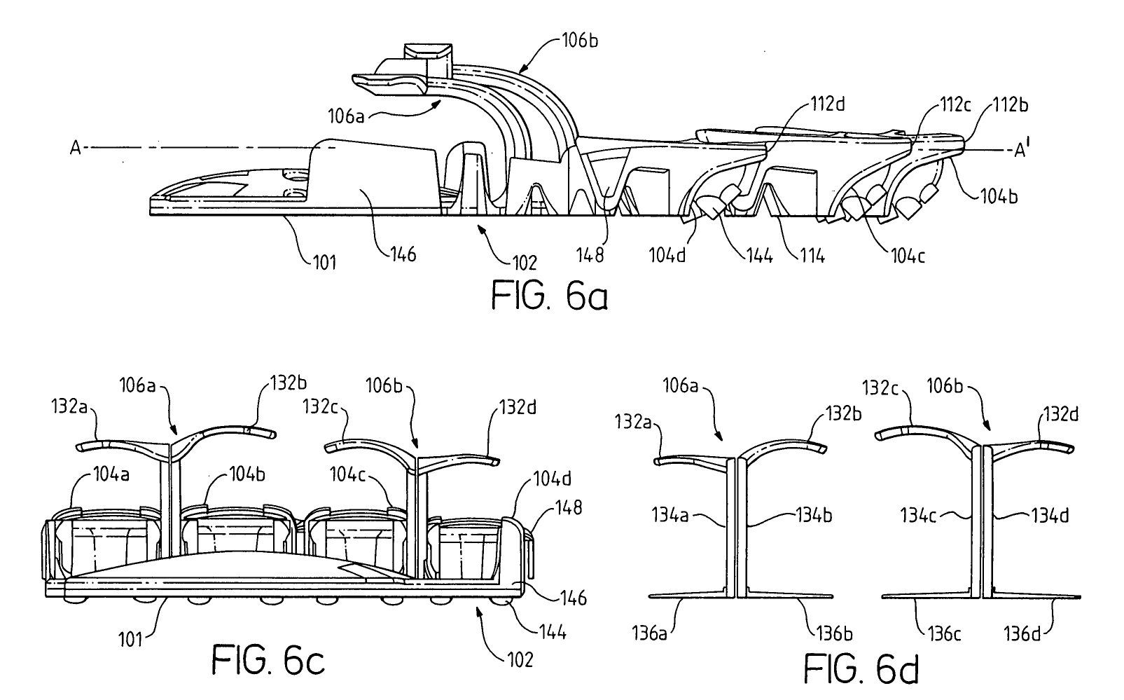

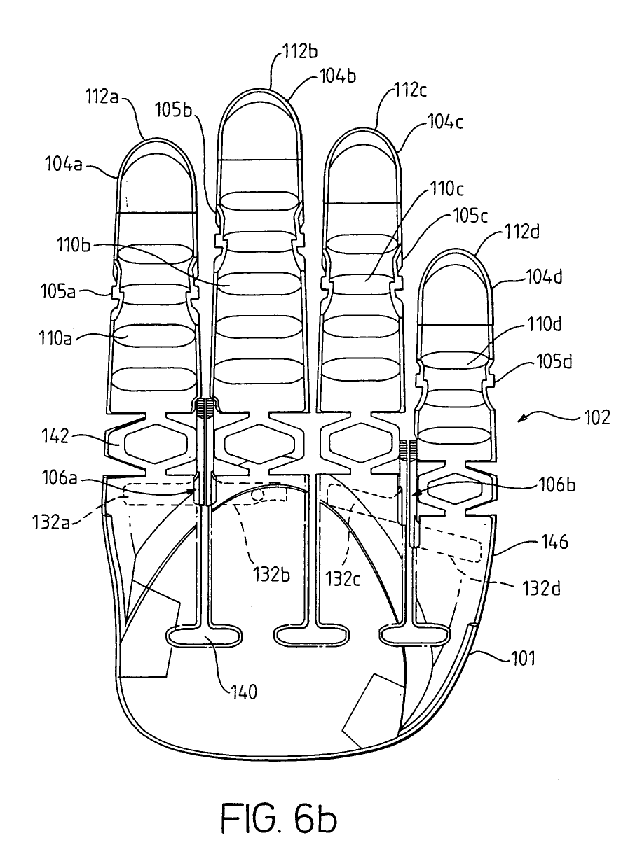

FIGS. 6a-6c illustrate an alternative glove 102 herein and FIG. 6d illustrates palm support means 106a, 106b suitable for use with the glove of FIGS. 6a-6c. Each finger portion 110a-110d of the glove 102 is provided with a finger grip 104a-104d in the form of a cage, which surrounds the end of a user's finger and each of which is provided with a thimble-like end 112a-112d. Each finger 110a-110d of the glove 102 is also provided with an inwardly tapering constriction 105a-105d located at a point below the foremost portion of the finger tip cage 104a-104d, which in use, corresponds approximately to a point below the first joint of the user's finger. The gripping portion of the finger tip cage 104a-d (i.e. that which acts on the constriction 105a-d) is that part interior to the lug 148, as shown in FIG. 6a. The gripping point will differ for different user hand sizes. On a small hand, the constriction 105a-105d locates more or less central to the first and second finger joints; on a large hand the constriction 105a-105d locates just behind the first finger joint. It may be appreciated that in use, both the finger grip framework 104a-104d and the constriction 105a-105d act such as to retain the user's finger within the glove.

The glove 102 is also provided with T-form yokes 106a, 106b each comprising pairs of gripping arms 132a, 132b and 132c, 132d (shown in outline in FIG. 6a). The form of the T-form yokes 106a, 106b may be better understood by reference to FIG. 6d from which it may be appreciated that each gripping arm 132a-132d is independently movable. Indeed, each yoke 106a, 106b is comprised of two separate arm 132a, 132c and 132b, 132d; trunk 134a, 134c and 134b, 134d; and base 136a, 136c and 136b, 136d elements. In use, the T-forms yokes 106a, 106b respectively engage the node between a user's first and second, and third and fourth fingers.

In tandem, the finger grips 104a-104d and T-form yokes 106a, 106b form the means to retain the glove on the user's hand. The independent movability of each of these elements, in turn contributes to the ability of the glove to both protect the user's palm and fingers without hampering the movement of the user's hand and fingers.

The underside base 101 of the glove 102 is formed from a flexible, but resilient material (e.g. foam). The base 101 is provided with various shape characteristics to maximise its flexibility. These comprise longitudinal cutaway portions 140 (one only labelled, for clarity) in the palm of base 101; diamond-shaped extensible portions 142 (again, one only labelled) at the palm-end of each finger portion 110a-110d, which provide flexibility in accommodating an unusually long user's finger; and “v”-shaped incisions 114 (one labelled only) running transversely across the underside of each finger portion 110a-110d of the glove 102, which enables ready finger movement.

The base 101 of the glove 102 is also provided at the fingers 110a-110d with angled bristle stocks 144 (one only labelled, for clarity). In other embodiments, various utility attachments may also be provided to the base 101, as described herein before. The base 101 is further provided with wall 146, which assists the stability of the interface 102 on the user's hand when in use. Each finger grip 104a-d is further provided with substantially triangular-shaped lug 148 (one only shown, for clarity) for use in mounting the glove 102 to a base station or housing (e.g. as shown in FIG. 5).

In use, base 101 of the glove 102 is designed to cover at least that part of a user's palm that can be comfortably reached by the user's fingers once they are curled over. This ensures that absorbent cleaning media can be efficiently wrung out by a simple clenching of the user's fist. Other types of media may be attached to other parts of the glove 102 and extend to the periphery of the glove body 102 and/or to the area covered by the whole hand.

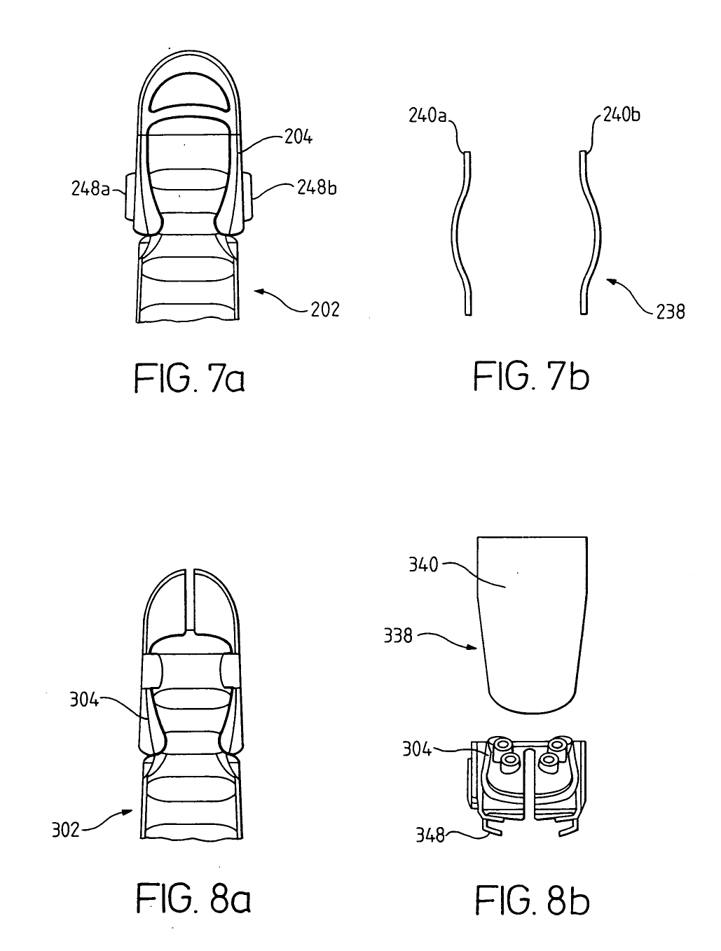

FIGS. 7a (which essentially shows a plan view of element 112d of FIG. 6a) and 7b show details of the ‘docking interaction’ between the finger grip means 204 of one finger of a glove-like interface 202 herein (FIG. 7a, shown in top view) with parking element 240a, 240b of a base station 238 (FIG. 7b, shown in plan view). The cage-like finger grip means 204 are provided with side mounted lugs 248a, 248b, which are shaped to cooperate with the parking elements 240a, 240b of the base station 238. In more detail, the parking elements 240a, 240b comprise outward bowed mounting rails such that, when a user's finger pushes the finger grip means 204 into the base station 238, the finger grips 204 are caused to open, so releasing the user's finger and “parking” the finger of the glove 202 in the railed parking elements 240a, 240b. In use, the glove 202 automatically couples to the user's hand once again by inserting a finger and driving it forward off the rails 240a, 240b.

FIGS. 8a and 8b show details of another type of ‘docking interaction’ between the finger grip means 304 of one finger of a glove-like interface 302 herein (top view in FIG. 8a; finger tip end elevation view in FIG. 8b) with tongue-like parking element 340 of a base station 338. The finger grip means 304 have a sprung cage structure which co-operates with top-mounted lug 348 (the ear-form elements of FIG. 8a) which is shaped to cooperate with the parking element 340 of the base station 338. In more detail, when a user's finger pushes the sprung cage 304 into the tapering tongue like parking element 340 of the base station 338, the sprung finger grips 304 are caused to open, so releasing the user's finger and “parking” the finger of the glove 302. In use, the glove 302 automatically couples to the user's hand once again by inserting a finger into the caged structure 304 and driving it off the parking tongue 340.

It may be appreciated that gloves herein may be configured to enable the docking-type interactions of FIGS. 7a-7b and 8a-8c to occur for one or many of the fingers of the relevant glove. Suitably, all fingers of the glove may be parked in the base station, which has parking elements appropriately spaced to ensure ease of glove-base station interaction.

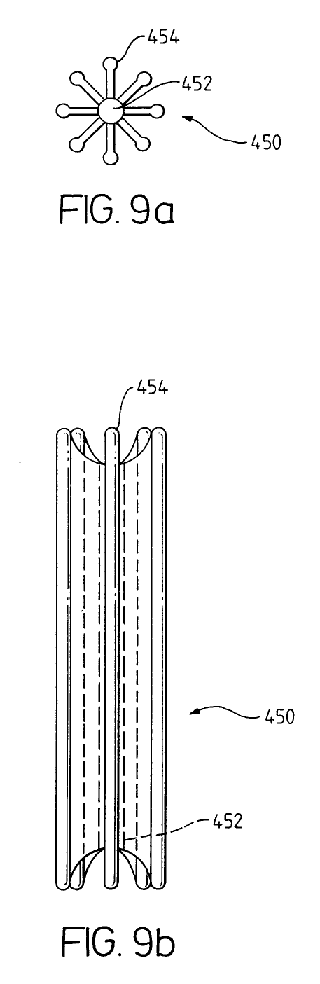

FIGS. 9a and 9b show views of a finned bar 450 shaped for grip by the clenched fist of a glove herein (not shown in FIG. 9). The finned bar 450 has plural fins 454 (one only labelled, for clarity) mounted on a central bar 452. In use, a gloved user's hand will clench the finned bar 450, which enables the better wringing out of the glove.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. A glove-like interface for use in protecting a user's hand during utility tasks comprising
a body having a plurality of finger members extending therefrom; and
resilient hand support means for securing the glove to a hand,
wherein said resilient hand support means comprises both finger support means and palm support means for respectively securing the glove to the fingers and palm of said user's hand.

2. A glove-like interface according to claim 1, in the form of a part-glove.

3. A glove-like interface according to either of claims 1 or 2, having an open framework or lattice-type structure.

4. A glove-like interface according to any of claims 1 to 3, which in use, covers the users' fingers and at least part of the user's palm but leaves the top part of the user's hand and fingers uncovered.

5. A glove-like interface according to any of claims 1 to 4, wherein in use, the body covers part or all of the user's palm and fingers.

6. A glove-like interface according to any of claims 1 to 5, wherein the body is shaped to provide a flat work surface.

7. A glove-like interface according to any of claims 1 to 6, wherein the body is provided with one or more cuts to improve the flexibility thereof.

8. A glove-like interface according to claim 7, wherein a “T-form” cut is provided to a palm element of the body on a line between each finger member.

9. A glove-like interface according to any of claims 1 to 8, wherein each finger member incorporates a feature at the junction thereof with a palm element of the body, which flexes to account for extremes of user's finger length.

10. A glove-like interface according to claim 9, wherein said feature comprises a diamond-shaped extendable element.

11. A glove-like interface according to any of claims 1 to 10, wherein one or more of the finger members incorporates a side wall to at least partially encase a user finger.

12. A glove-like interface according to any of claims 1 to 11, additionally comprising an ancillary interface.

13. A glove-like interface according to any of claims 1 to 12, wherein both the finger support means and the palm support means have a gripping function.

14. A glove-like interface according to any of claims 1 to 13, wherein the resilient finger support means acts to hold the glove tight against the underside of at least part of the user's fingers.

15. A glove-like interface according to any of claims 1 to 14, wherein the resilient finger support means acts to hold the glove tight against the user's finger-tips.

16. A glove-like interface according to any of claims 1 to 15, wherein the resilient finger support means comprises a caged framework shaped to snugly receive a user's finger tip.

17. A glove-like interface according to any of claims 1 to 16, wherein the resilient finger support means defines a constricted passageway shaped to engage with a user's finger.

18. A glove-like interface according to any of claims 1 to 17, wherein the resilient finger support means takes the form of a sprung clip.

19. A glove-like interface according to any of claims 1 to 18, wherein the resilient finger support means are provided with side or top mounted lugs, which are shaped to cooperate with a base station.

20. A glove-like interface according to any of claims 1 to 19, wherein the palm support means comprise one or more resilient yokes.

21. A glove-like interface according to claim 20, wherein the one or more resilient yokes have a T-form.

22. A glove-like interface according to either of claims 20 or 21, wherein each yoke comprises a vertical trunk with horizontally extending arms and legs.

23. A glove-like interface according to any of claims 20 to 22, wherein each yoke comprises two pairs of independent arms, each pair mounted on a vertical trunk.

24. A glove-like interface according to any of claims 1 to 23, comprising two or three resilient palm support means, each of which comprises a pair of independent arms.

25. A glove-like interface according to any of claims 1 to 24, wherein the resilient finger and/or palm support means are made from sprung steel wire or moulded plastics.

26. A glove-like interface according to any of claims 1 to 25, wherein each finger member comprises at least one substantially “V”-shaped excision running transversely across the underside thereof.

27. A glove-like interface according to claim 26, wherein a bridge member is provided between each “V”-shaped excision.

28. A glove-like interface according to any of claims 1 to 27, formed from a semi-rigid or rigid material.

29. A glove-like interface according to any of claims 1 to 28, additionally comprises attachment means for attaching one or more utility attachments.

30. A glove-like interface according to claim 29, wherein the one or more utility attachments comprise one or more tools.

31. A glove-like interface according to either of claims 29 or 30, wherein the utility attachments are permanently attachable to the glove.

32. A glove-like interface according to either of claims 29 or 30, wherein the utility attachments are reversibly receivable by the glove.

33. A glove-like interface according to any of claims 29 to 32, wherein the utility attachments take the form of bristles, spines, hooks, hair, sponge, leather, fabric pads, scourers, abrasives or wire wool.

34. A glove-like interface according to any of claims 29 to 33, wherein the utility attachments comprise vacuum elements.

35. A glove-like interface according to any of claims 29 to 34, wherein the utility attachments are positioned at the front and/or underside and/or sides of said finger members.

36. A glove-like interface according to any of claims 29 to 34, wherein the utility attachments are receivable by the body of the glove.

37. A glove-like interface according to any of claims 1 to 36, additionally incorporating flows means for flowing liquids and gasses thereto.

38. Kit of parts herein comprising a glove-like interface according to any of claims 1 to 37 and a bar having fin elements provided thereto, wherein squeezing the bar in the fist of the glove-like interface allows effective wringing out thereof.

39. Kit of parts herein comprising a glove-like interface according to any of claims 1 to 37 and a base station therefor, wherein one or more of the finger support means of the glove-like interface is provided with one or more lugs arranged to co-operate with one or more lug-receivers of the base station.

40. Kit of parts according to claim 39, wherein said one or more lug-receivers of the base station each comprises a pair of bowed mounting rails.

41. Kit of parts herein comprising a glove-like interface according to any of claims 1 to 37 and a base station therefor, wherein one or more of the finger support means of the glove-like interface is in the form of a flexible cage arranged to co-operate with one or more cage-opening elements of the base station such that each flexible cage is flexed open on interaction with a cage-opening element of the base station.
